(19) United States
(12) Patent Application Publication
(10) Pub. No.: US 2026/0102211 A1
(43) Pub. Date: Apr. 16, 2026

(54) METHOD AND APPARATUS FOR GENERATING MEDICAL PREDICTION OF ATRIAL FIBRILLATION RECURRENCE AFTER ABLATION

(71) Applicants: Case Western Reserve University, Cleveland, OH (US); Emory University, Atlanta, GA (US); Vanderbilt University, Nashville, TN (US); The Cleveland Clinic Foundation, Cleveland, OH (US)

(72) Inventors: Golnoush Asaeikheybari, Cleveland Heights, OH (US); Anant Madabhushi, Decatur, GA (US); Majd El-Harasis, Atlanta, GA (US); Moore Benjamin Shoemaker, Atlanta, GA (US); John Barnard, Cleveland, OH (US); Mina K. Chung, Shaker Heights, OH (US)

(21) Appl. No.: 19/355,117

(22) Filed: Oct. 10, 2025

Related U.S. Application Data
(60) Provisional application No. 63/706,366, filed on Oct. 11, 2024.

(51) Int. Cl. A61B 34/10; A61B 18/00; A61B 18/12; G06T 7/00

(52) U.S. Cl. CPC A61B 34/10; A61B 18/12; G06T 7/0014; A61B 2018/00351; A61B 2018/00577; A61B 2018/00845; A61B 2034/104; A61B 2034/105; G06T 2207/30048

(57) ABSTRACT

The present disclosure, in some embodiments, relates to a method that includes accessing digitized imaging data stored in an electronic memory. The digitized imaging data comprises one or more regions of interest including pulmonary vein branches of a patient. A plurality of radiomic features are extracted from the one or more regions of interest. The plurality of radiomic features include one or more of fractal-based features or mesh-based features. The plurality of radiomic features are provided to a machine learning stage. The machine learning stage is configured to utilize the plurality of radiomic features to generate a medical prediction corresponding to atrial fibrillation recurrence after an ablation procedure.

### REFERENCE TO RELATED APPLICATION

This Application claims the benefit of U.S. Provisional Application No. 63/706,366, filed on Oct. 11, 2024, the contents of which are incorporated by reference in their entirety.

### GOVERNMENT SUPPORT CLAUSE

This invention was made with government support under HL111314, HL090620, and HL158502 awarded by the National Institutes of Health. The government has certain rights in the invention.

### BACKGROUND

Atrial fibrillation is a common heart rhythm disorder that is characterized by an irregular and often very rapid heartbeat. Atrial fibrillation can lead to an increased risk of blood clots in the heart, a heart attack, a stroke, heart failure, and/or other heart-related complications. There are different types of atrial fibrillation. For example, paroxysmal atrial fibrillation is a type of arrhythmia that comes and goes, while persistent atrial fibrillation is a type of arrhythmia that last for more than seven days. While atrial fibrillation usually isn't life-threatening, it is a serious medical condition that should get proper treatment.

### BRIEF DESCRIPTION OF THE DRAWINGS

The accompanying drawings, which are incorporated in and constitute a part of the specification, illustrate various example operations, apparatus, methods, and other example embodiments of various aspects discussed herein. It will be appreciated that the illustrated element boundaries (e.g., boxes, groups of boxes, or other shapes) in the figures represent one example of the boundaries. One of ordinary skill in the art will appreciate that, in some examples, one element can be designed as multiple elements or that multiple elements can be designed as one element. In some examples, an element shown as an internal component of another element may be implemented as an external component and vice versa. Furthermore, elements may not be drawn to scale.

FIG. 1 illustrates some embodiments of an atrial fibrillation recurrence identification system configured to use radiomic features extracted from pulmonary vein branches to generate a medical prediction corresponding to atrial fibrillation recurrence.

FIG. 2 illustrates some embodiments of a method of using radiomic features extracted from pulmonary vein branches to generate a medical prediction corresponding to atrial fibrillation recurrence.

FIG. 3 illustrates some additional embodiments of an atrial fibrillation recurrence identification system configured to use radiomic features extracted from pulmonary vein branches.

FIG. 4 illustrates some additional embodiments of an atrial fibrillation recurrence identification system configured to use radiomic features extracted from pulmonary vein branches.

FIG. 5A illustrates some embodiments of a disclosed segmentation process configured to identify pulmonary vein branches within digitized imaging data.

FIG. 5B illustrates some additional embodiments of a disclosed segmentation process configured to identify pulmonary vein branches within digitized imaging data.

FIG. 5C illustrates some embodiments of digitized images showing a segmented left atrium and pulmonary vein branches.

FIG. 6A illustrates a table showing exemplary radiomic features that may be used to generate a medical prediction corresponding to atrial fibrillation.

FIG. 6B illustrates some embodiments of exemplary segmented images and corresponding heat maps illustrating 3D fractal dimensions.

FIG. 6C illustrates some embodiments of violin plots of exemplary radiomic features extracted from primary pulmonary vein branches.

FIG. 7 illustrates some additional embodiments of an atrial fibrillation recurrence identification system configured to use radiomic features to generate a medical prediction corresponding to atrial fibrillation recurrence.

FIG. 8A illustrates some additional embodiments of an atrial fibrillation recurrence identification system configured to use radiomic features to generate a medical prediction corresponding to atrial fibrillation recurrence.

FIG. 8B illustrates a table showing performance parameters of different machine learning models used in the atrial fibrillation recurrence identification system of FIG. 8A.

FIG. 9 illustrates a flow diagram showing some additional embodiments of a method of operating a machine learning stage on radiomic features to generate a medical prediction corresponding to atrial fibrillation recurrence.

FIG. 10 illustrates some embodiments of a block diagram of a disclosed atrial fibrillation recurrence identification system.

### DETAILED DESCRIPTION

The description herein is made with reference to the drawings, wherein like reference numerals are generally utilized to refer to like elements throughout, and wherein the various structures are not necessarily drawn to scale. In the following description, for purposes of explanation, numerous specific details are set forth in order to facilitate understanding. It may be evident, however, to one of ordinary skill in the art, that one or more aspects described herein may be practiced with a lesser degree of these specific details. In other instances, known structures and devices are shown in block diagram form to facilitate understanding.

Atrial fibrillation is one of the most frequently occurring types of heart arrhythmia. It is associated with an increased risk of blood clots, stroke, heart failure, etc. An ablation (e.g., a catheter ablation) is a medical procedure that is commonly used to treat atrial fibrillation. During an ablation, a doctor will identify one or more areas within a patient's heart that are causing an abnormal heart rhythm. The doctor will then selectively destroy or “ablate” tissue within the one or more areas (e.g., using heat or cold). Once the tissue is destroyed, the abnormal electrical signals that caused the arrhythmia can no longer be sent to the rest of the heart, thereby mitigating the arrhythmia.

However, within one year of an ablation procedure atrial fibrillation will recur among approximately 20% to approximately 40% of patients. Despite substantial research aimed at trying to predict atrial fibrillation recurrence, there is no known method for reliably predicting risk and/or sites associated with atrial fibrillation recurrence. For example, while pulmonary vein size and volume have been associated with atrial fibrillation recurrence, the association is not strong enough to be used as a factor in patient selection or ablation targeting. Being able to accurately identify a risk of atrial fibrillation recurrence is important to health care professionals and/or patients considering an ablation procedure. Furthermore, being able to identify anatomical sites associated with atrial fibrillation recurrence is critical to help ablation procedures to minimize a likelihood of atrial fibrillation recurrence.

The present disclosure relates to a method and apparatus that uses radiomic features extracted from pulmonary vein branches to generate a medical prediction corresponding to atrial fibrillation recurrence. In some embodiments, the method includes accessing digitized imaging data stored in an electronic memory. The digitized imaging data may be segmented to identify one or more regions of interest (ROIs) including pulmonary vein branches of a patient. A plurality of radiomic features are extracted from the one or more ROIs. The plurality of radiomic features characterize a structural complexity and/or roughness of the pulmonary vein branches. The plurality of radiomic features are provided to a machine learning stage, which is configured to utilize the plurality of radiomic features to generate a medical prediction corresponding to atrial fibrillation recurrence after an ablation procedure. It has been appreciated that there is a correlation between the morphology of pulmonary vein branches and atrial fibrillation recurrence. Because there is this correlation, the disclosed method is able to generate the medical prediction relating to atrial fibrillation recurrence with a high degree of accuracy that can improve outcomes for atrial fibrillation patients.

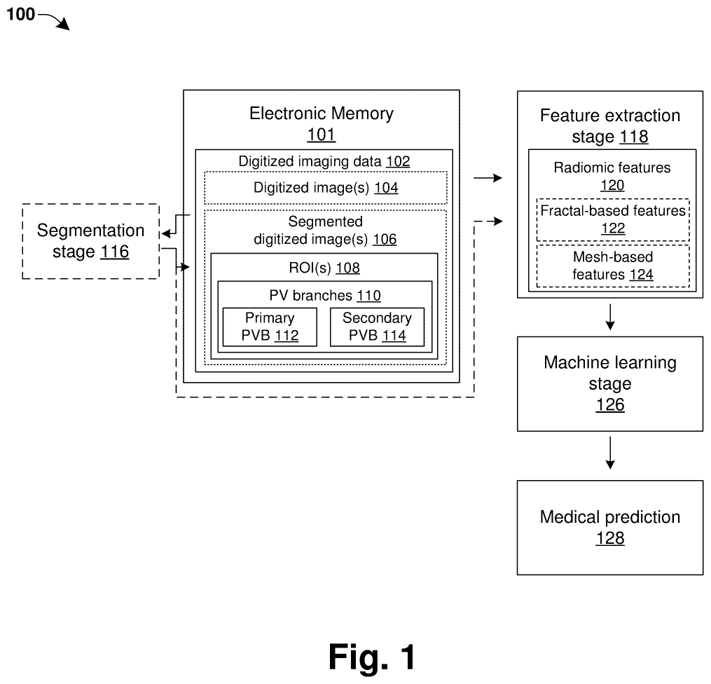

FIG. 1 illustrates some embodiments of an atrial fibrillation recurrence identification system 100 configured to use radiomic features extracted from pulmonary vein branches to generate a medical prediction corresponding to atrial fibrillation recurrence.

The atrial fibrillation recurrence identification system 100 comprises electronic memory 101 configured to store digitized imaging data 102 from a patient. The digitized imaging data 102 may comprise one or more digitized images 104. In various embodiments, the digitized imaging data 102 may include pre-ablation imaging data (e.g., imaging data from one or more patients that may subsequently undergo an ablation) and/or post ablation imaging data (e.g., imaging data from one or more patients that have undergone an ablation). In some embodiments, the digitized imaging data 102 may comprise computer tomography (CT) images, magnetic resonance imaging (MRI) images, and/or the like. In some embodiments, the digitized imaging data 102 may comprise segmented digitized images 106 that identify one or more regions of interest (ROIs) 108 including a plurality of pulmonary vein branches 110. In various embodiments, the plurality of pulmonary vein branches 110 may comprise one or more of primary pulmonary vein branches 112 and/or secondary pulmonary vein branches 114.

In some embodiments, a segmentation stage 116 may be in communication with the electronic memory 101. The segmentation stage 116 is configured to generate the segmented digitized images 106 by identifying the one or more ROIs 108 (e.g., the plurality of pulmonary vein branches 110) within the one or more digitized images 104. In some embodiments, the segmentation stage 116 may comprise one or more deep learning models. In some embodiments, the segmentation stage 116 may comprise one or more convolutional neural networks (CNNs) (e.g., having a U-Net architecture).

In some embodiments, a feature extraction stage 118 is configured to extract a plurality of radiomic features 120 from the one or more ROIs 108 (e.g., from one or more of the primary pulmonary vein branches 112 and/or the secondary pulmonary vein branches 114). In some embodiments, the plurality of radiomic features 120 characterize morphological attributes associated with pulmonary vein structures. The morphological attributes may include fine-scale details, topographical variations of pulmonary vein surfaces (e.g., roughness), and/or the like. In some embodiments, the plurality of radiomic features 120 may comprise one or more of fractal-based features 122 and/or mesh-based features 124. The fractal-based features 122 comprise features that are generated using fractal geometry (e.g., spatial pattern self-similarity) to characterize tissue structures (e.g., including complex and irregular tissue structures). The mesh-based features 124 comprise features that may characterize surface roughness by quantifying deviations or irregularities on a surface.

The plurality of radiomic features 120 are provided to a machine learning stage 126 comprising one or more machine learning models. The machine learning stage 126 is configured to utilize the plurality of radiomic features 120 to generate a medical prediction 128 corresponding to atrial fibrillation recurrence (e.g., recurrence of atrial fibrillation within approximately 3 to 12 months after an ablation procedure) within a patient. It has been appreciated that there may be a direct correlation between recurrence of atrial fibrillation and a high level of surface complexity (e.g., higher fractal dimension values compared to cases experiencing non-recurrence of atrial fibrillation) in pulmonary vein branches. Because the medical prediction 128 corresponding to atrial fibrillation recurrence utilizes radiomic features that characterize a morphology of pulmonary vein branches, the medical prediction 128 can account for surface complexity, pulmonary vein remodeling (e.g., structural changes to the walls of the pulmonary veins), and/or the like, to achieve a high degree of accuracy that improves patient care.

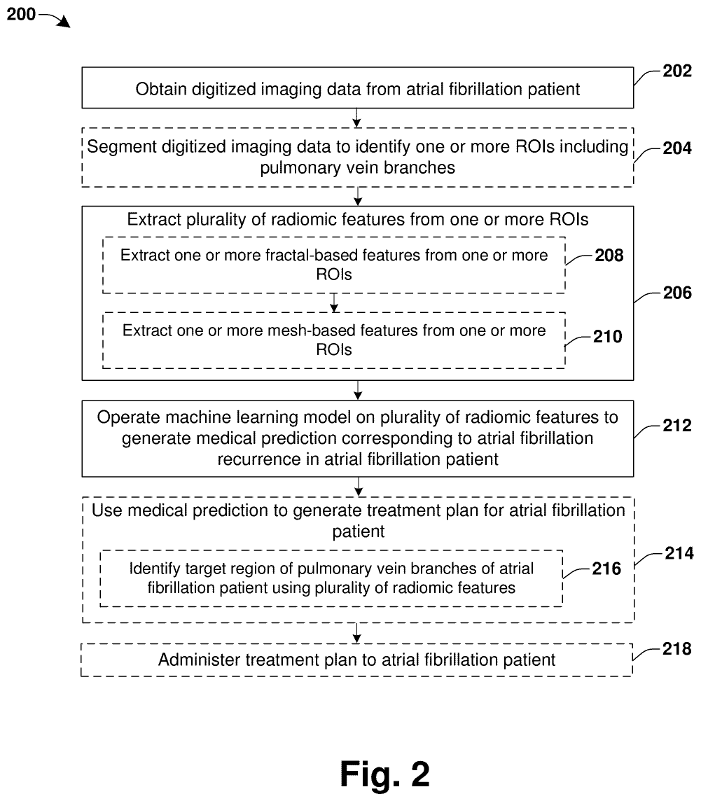

FIG. 2 illustrates some embodiments of a method 200 of using radiomic features extracted from pulmonary vein branches to generate a medical prediction corresponding to atrial fibrillation recurrence.

While the disclosed methods (e.g., methods 200 and 900) are illustrated and described herein as a series of acts or events, it will be appreciated that the illustrated ordering of such acts or events are not to be interpreted in a limiting sense. For example, some acts may occur in different orders and/or concurrently with other acts or events apart from those illustrated and/or described herein. In addition, not all illustrated acts may be required to implement one or more aspects or embodiments of the description herein. Further, one or more of the acts depicted herein may be carried out in one or more separate acts and/or phases.

At act 202, digitized imaging data is obtained from an atrial fibrillation patient. In some embodiments, the digitized imaging data may comprise one or more digitized images (e.g., one or more CT images). In some embodiments, the digitized imaging data may be obtained by operating an imaging tool (e.g., a CT scanner) on the atrial fibrillation patient. In other embodiments, the digitized imaging data may be obtained by accessing imaging data stored in electronic memory.

At act 204, the digitized imaging data may be segmented to identify one or more ROIs including pulmonary vein branches in some embodiments. In some embodiments, the one or more ROIs may include primary and/or secondary pulmonary vein branches.

At act 206, a plurality of radiomic features are extracted from the one or more ROIs. The plurality of radiomic features may characterize morphological attributes associated with pulmonary vein branch structures. In some embodiments, the plurality of radiomic features may characterize morphological attributes associated with a primary pulmonary vein branch and/or a secondary pulmonary vein branch. In some embodiments, the plurality of radiomic features may be extracted from the digitized imaging data according to acts 208-210.

At act 208, one or more fractal-based features are extracted from the one or more ROIs. The one or more fractal-based features may mathematically characterize a geometrical complexity of a tissue structure by using spatial pattern self-similarity.

At act 210, one or more mesh-based features are extracted from the one or more ROIs. The one or more mesh-based features mathematically quantify small-scale deviations or irregularities on a tissue surface.

At act 212, a machine learning model is operated on the plurality of radiomic features to generate a medical prediction corresponding to atrial fibrillation recurrence in the atrial fibrillation patient.

At act 214, the medical prediction may be used to generate a treatment plan for the atrial fibrillation patient. The treatment plan may include application of drugs (e.g., beta-blockers, flecainide, propafenone, amiodarone, etc.), medical procedures (e.g., repeating catheter ablation, electrical cardioversion, etc.), making lifestyle changes to manage underlying risk factors like high blood pressure and obesity, etc. In some embodiments, the treatment plan may be generated according to act 216.

At act 216, a target region of pulmonary vein branches may be identified using the plurality of radiomic features. In some embodiments, the target region may comprise a physical part of the pulmonary vein branches that may be identified as being associated with atrial fibrillation recurrence in the atrial fibrillation patient.

At act 218, the treatment plan may be administered to the atrial fibrillation patient by a health care professional. In some embodiments, the treatment plan may be administered by performing an ablation procedure (e.g., a radiofrequency catheter ablation process, a catheter cryoablation process, etc.) that targets the target region of the atrial fibrillation patient.

It will be appreciated that the disclosed methods and/or block diagrams may be implemented as computer-executable instructions, in some embodiments. Thus, in one example, a computer-readable storage device (e.g., a non-transitory computer-readable medium) may store computer executable instructions that if executed by a machine (e.g., computer, processor) cause the machine to perform the disclosed methods and/or block diagrams. While executable instructions associated with the disclosed methods and/or block diagrams are described as being stored on a computer-readable storage device, it is to be appreciated that executable instructions associated with other example disclosed methods and/or block diagrams described or claimed herein may also be stored on a computer-readable storage device.

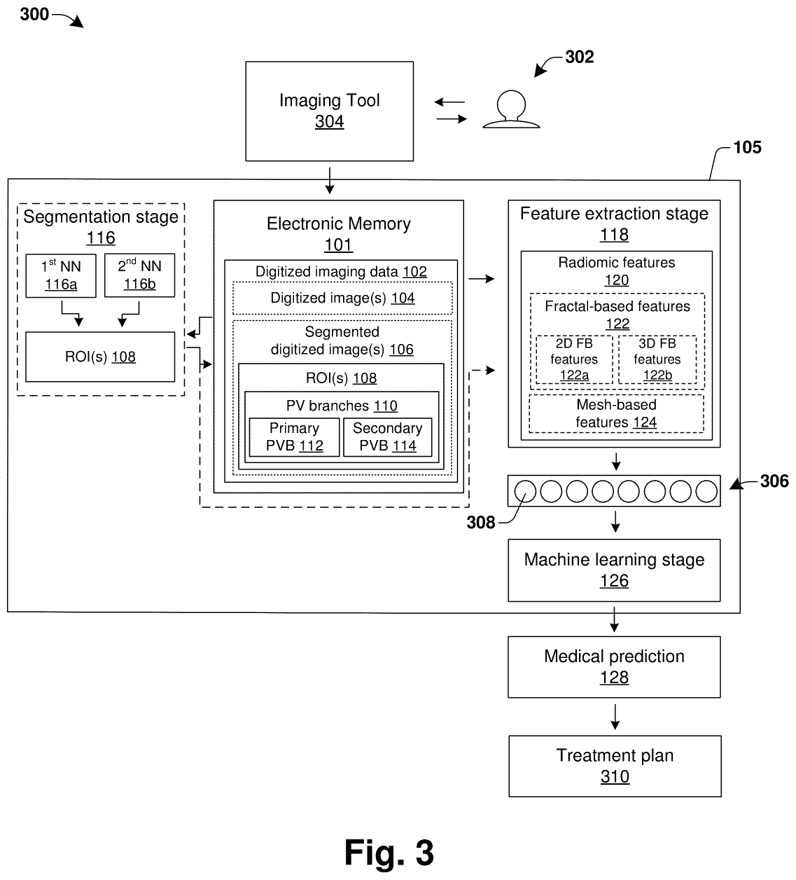

FIG. 3 illustrates some additional embodiments of an atrial fibrillation recurrence identification system 300 configured to use radiomic features extracted from pulmonary vein branches.

The atrial fibrillation recurrence identification system 300 comprises an atrial fibrillation recurrence identification tool 105 comprising electronic memory 101 configured to store digitized imaging data 102 from a patient 302. The digitized imaging data 102 may comprise one or more digitized images 104 (e.g., one or more pre-ablation and/or post ablation CT images). In some embodiments, the digitized imaging data 102 may be obtained from an imaging tool 304 that is configured to operate upon the patient 302. The imaging tool 304 may comprise a CT machine with an integrating detector, an energy sensitive CT machine (e.g., using multiple types of implementations), a photon-counting CT machine, an MRI machine, and/or the like. In some embodiments, the digitized imaging data 102 may comprise one or more segmented digitized images 106 that identify one or more regions of interest (ROIs) 108 including a plurality of pulmonary vein branches 110.

In some embodiments, a segmentation stage 116 is in communication with the electronic memory 101. The segmentation stage 116 is configured to form the one or more segmented digitized images 106 that identify the one or more ROIs 108 within the one or more digitized images 104. In various embodiments, the one or more ROIs 108 may comprise one or more of primary pulmonary vein branches 112 (e.g., including superior and/or inferior pulmonary vein branches extending outward from a heart and/or from a left atrium of a heart) and/or secondary pulmonary vein branches 114 (e.g., including smaller vessels that extend outward from the superior pulmonary vein branches). In some embodiments, the primary pulmonary vein branches 112 may include a right superior pulmonary vein, a right inferior pulmonary vein, a left superior pulmonary vein, and/or a left inferior pulmonary vein.

In some embodiments, the segmentation stage 116 is configured to perform a probabilistic image segmentation to generate one or more probability maps (e.g., a grayscale image where each pixel's value represents a probability that a corresponding pixel in an original image belongs to a specific object). In some embodiments, the segmentation stage 116 may convert the one or more probability maps into binary masks. In some such embodiments, the one or more binary masks comprise images having a value of “1” in image units (e.g., pixels, voxels, etc.) identified as being within the primary pulmonary vein branches 112 and/or the secondary pulmonary vein branches 114 and having a value of “0” in image units outside of the primary and/or secondary pulmonary vein branches.

In some embodiments, the segmentation stage 116 may comprise one or more deep learning models, convolutional neural networks (CNNs), or the like. In some embodiments, the segmentation stage 116 may comprise an artificial intelligence based segmentation pipeline comprising a plurality of different steps. The artificial intelligence based segmentation pipeline comprising a plurality of different steps enables the segmentation stage 116 to accurately segment complex branching patterns of pulmonary vasculatures and account for inter-reader variability (e.g., differences in digitized images between scanners and/or health care institutions) in light of pulmonary vein complications due to proximity to other pulmonary structures and variable presentations across individuals.

In some embodiments, the segmentation stage 116 may comprise a plurality of segmentation models 116a-116b configured to collectively identify the one or more ROIs 108. In some embodiments, the plurality of segmentation models 116a-116b may comprise neural networks that have different resolutions. For example, the plurality of segmentation models 116a-116b may comprise a first neural network 116a having a first resolution (e.g., a U-Net having a first resolution) and a second neural network 116b having a second resolution (e.g., a U-Net having a second resolution) that is less than the first resolution. The first neural network 116a and the second neural network 116b may separately generate probability maps that are combined to identify the one or more ROIs 108.

A feature extraction stage 118 is configured to extract a plurality of radiomic features 120 from the one or more ROIs 108. In some embodiments, the plurality of radiomic features 120 characterize morphological attributes associated with pulmonary vein branch structures. In some embodiments, the plurality of radiomic features 120 may comprise fractal-based features 122 and mesh-based features 124.

The plurality of radiomic features 120 are provided as input data 306 (e.g., as a 1-dimensional vector or a multi-dimensional matrix) to a machine learning stage 126 comprising one or more machine learning models. The machine learning stage 126 is configured to generate a medical prediction 128 by operating upon the input data 306 to determine weightings associated with values 308 of the input data 306. The weightings assign different levels of importance to various radiomic features within the input data 306, thereby influencing their impact on the medical prediction 128.

In some embodiments, the medical prediction 128 may be utilized to generate a treatment plan 310 for the patient 302. In contrast to deep-learning approaches, which provide “black box” predictions, the plurality of radiomic features 120 are more biologically interpretable due to their description of the shape of the pulmonary vein branches. This higher interpretability may allow for the medical prediction 128 to be used to identify precise anatomical characteristics that might contribute to and/or serve as indicators of atrial fibrillation recurrence. These precise anatomical characteristics may be used to generate new anatomic targets and/or modifications for subsequent ablation procedures that may decrease a chance of atrial fibrillation recurrence. In addition, using the plurality of radiomic features 120 is computationally simpler and faster than deep learning training (which uses millions of parameters).

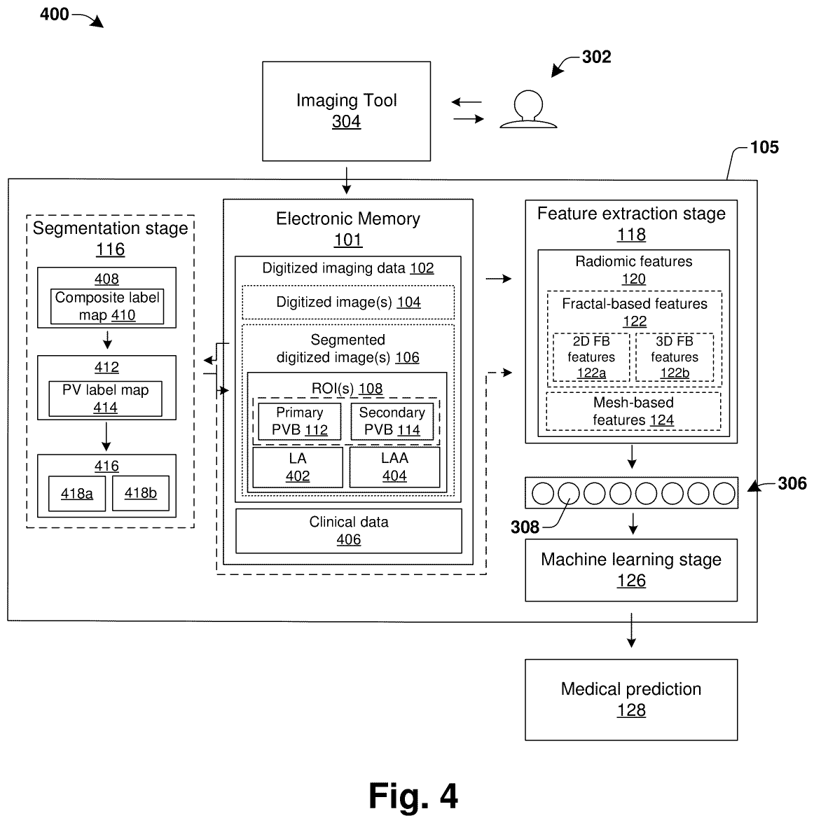

FIG. 4 illustrates some additional embodiments of an atrial fibrillation recurrence identification system 400 configured to use radiomic features extracted from pulmonary vein branches.

The atrial fibrillation recurrence identification system 400 comprises digitized imaging data from a patient 302. In some embodiments, the digitized imaging data 102 may comprise one or more digitized images 104 generated by an imaging tool 304 and stored in electronic memory 101. In various embodiments, the electronic memory 101 may comprise read-only memory (ROM), random-access memory (RAM), erasable programmable read-only memory (EPROM), electrically erasable programmable read-only memory (EEPROM), flash memory, dynamic random-access memory (DRAM), static random-access memory (SRAM), and/or the like.

In some embodiments, the digitized imaging data 102 may comprise one or more segmented digitized images 106 that identify one or more regions of interest (ROIs) 108 within the one or more digitized images 104. The one or more ROIs 108 may comprise a plurality of pulmonary vein branches 110. The plurality of pulmonary vein branches 110 include primary pulmonary vein branches 112 and/or secondary pulmonary vein branches 114. In some embodiments, the one or more ROIs 108 may comprise one or more of a left atrium 402, a left atrial appendage 404, a combination of the primary pulmonary vein branches 112 and secondary pulmonary vein branches 114, and/or the like. In some embodiments, the one or more ROIs 108 may comprise the primary pulmonary vein branches 112, but not secondary pulmonary vein branches 114. In some embodiments, the one or more ROIs 108 may comprise primary pulmonary vein branches 112, but not the left atrium 402 and/or the left atrial appendage 404.

In some embodiments, a segmentation stage 116 is configured to generate the one or more segmented digitized images 106. In some embodiments, the segmentation stage 116 may comprise a plurality of neural networks. In some embodiments, the segmentation stage 116 may comprise one or more processors (e.g., a central processing unit including one or more transistor devices configured to operate computer code to achieve a result, a microcontroller, a graphics processing unit, and/or the like).

In some embodiments, the plurality of neural networks may be configured to collectively perform a first segmentation operation 408 that forms a composite label map 410 that identifies both a left atrium and the primary and secondary pulmonary vein branches. The plurality of neural networks may be further configured to perform a second segmentation operation 412 that identifies the left atrium 402. The segmentation stage 116 is configured to subtract the left atrium from the composite label map 410 to generate a pulmonary vein label map 414 that isolates the pulmonary veins in the cardiac anatomy. The segmentation stage 116 is further configured to perform morphologic operations 416 to remove secondary pulmonary vein branches from the pulmonary vein label map 414 to generate a primary pulmonary vein label map 418a that identifies the primary pulmonary vein branches 112. The segmentation stage 116 is further configured to remove primary pulmonary vein branches from the pulmonary vein label map 414 to generate a secondary pulmonary vein label map 418b that identifies the secondary pulmonary vein branches 114.

A feature extraction stage 118 is configured to extract a plurality of radiomic features 120 from the one or more ROIs 108. In some embodiments, the plurality of radiomic features 120 may comprise fractal-based features 122 and mesh-based features 124. In some embodiments, the plurality of radiomic features 120 may comprise first order statistics (e.g., mean, median, standard deviation, maximum, minimum, kurtosis, large-bin) taken over the one or more ROIs 108. In some embodiments, the feature extraction stage 118 may comprise one or more processors (e.g., a central processing unit including one or more transistor devices configured to operate computer code to achieve a result, a microcontroller, a graphics processing unit, and/or the like). In some embodiments, one or more of the plurality of radiomic features 120 may be imperceptible to the human eye. For example, the plurality of radiomic features 120 may comprise pixel intensity quantifications and/or frequency information that is imperceptible to the human eye.

The fractal-based features 122 may include two-dimensional (2D) fractal-based features 122a and/or three-dimensional (3D) fractal-based features 122b. In some embodiments, the fractal-based features 122 may be measured in both the spatial and frequency domains. In some embodiments, a box counting method (e.g., a conventional box counting method, a folding box counting method, and an overlapping box counting method) may be used for measuring the 2D and 3D fractal-based features in the spatial domain. In other embodiments, the fractal-based features in the spatial domain may be measured using a box counting method (e.g., with various methods for implementing box counting methods, such as conventional, folding, and overlapping, and/or the like). In some embodiments, the fractal-based features 122 in the frequency domain may be obtained via power spectral density analysis and a Fast Fourier Transform (FFT) algorithm. Power Spectral Density (PSD) analysis measures a distribution of a signal's power across different frequencies.

In some embodiments, the fractal-based features 122 may comprise a fractal dimension slope and a fractal dimension intercept in the spatial and the frequency domain calculated both in 2D and 3D. In some embodiments, the 2D fractal-based features may be obtained by applying the aforementioned methods over each slice of a binary segmentation of a segmented digitized image. Subsequently the mean, median, max, variance, skewness, and standard deviation of the fractal dimension and the fractal dimension intercept over multiple slices (e.g., over all slices) are calculated for the segmented digitized image. The 3D fractal-based features may be directly extracted from a 3D label map.

In some embodiments, the mesh-based features 124 may be generated by forming a mesh of connected polygons (e.g., triangles) over the one or more ROIs 108 (e.g., using a marching cubes algorithm, a marching cube Lewicki algorithm, etc.) and then computing the mesh-based features 124 from the mesh. The mesh-based features 124 may include mesh roughness from Gaussian Curvature, Difference of Normals (DON), and vertex local spatial density. The mesh-based features 124 may correspond to the mean, variance, standard deviation, and skewness of these features.

In some embodiments, different features may be extracted from a primary pulmonary vein and from a secondary pulmonary veins. The top most predictive radiomic features extracted from a primary pulmonary vein may comprise a mean of saliency map, mean of frequency 3D fractal intercept, skewness of spatial 2D fractal dimension, mean of frequency 2D fractal dimension, skewness of vertex spatial local density, mean and skewness of Difference of Normals (DON), maximum of frequency 3D fractal dimension, maximum of spatial 2D fractal dimension, skewness of saliency map, mean of frequency 3D fractal dimension, skewness of frequency 2D fractal dimension, skewness of frequency 3D fractal dimension, and/or skewness of frequency 2D fractal intercept. The top most predictive radiomic features extracted from a secondary pulmonary vein may comprise a standard deviation (std) of frequency 3D fractal dimension, std of frequency 3D fractal intercept, mean of saliency map, mean of DON, std of frequency 2D fractal dimension, skewness and std of saliency map, mean and maximum of frequency 3D fractal intercept, std of spatial 3D fractal dimension, skewness and maximum of spatial 2D fractal dimension, median of spatial 3D fractal dimension, and/or skewness of frequency 3D fractal dimension.

A machine learning stage 126 is configured to utilize the plurality of radiomic features 120 to generate a medical prediction 128 corresponding to atrial fibrillation recurrence. In some embodiments, the machine learning stage 126 may comprise a gradient boosting classifier. In other embodiments, the machine learning stage 126 may comprise a regression model, a Cox Hazard regression model, a support vector machine (SVM), a linear discriminant analysis (LDA) classifier, a Naïve Bayes classifier, a Random Forest, Adaboost, or the like. In some embodiments, the machine learning stage 126 may comprise one or more processors (e.g., a central processing unit including one or more transistor devices configured to operate computer code to achieve a result, a microcontroller, a graphics processing unit, and/or the like).

In some embodiments, the machine learning stage 126 may be further configured to utilize clinical data 406 of the patient 302 to generate the medical prediction 128. For example, clinical data 406 from the patient 302 may be stored in the electronic memory 101. The machine learning stage 126 may be configured to receive the clinical data 406 from the electronic memory 101 (e.g., as one or more values 308 of the input data 306). In some embodiments, the clinical data 406 may include an atrial fibrillation type (e.g., paroxysmal or persistent) type and/or a catheter ablation technique. It has been appreciated that the disclosed atrial fibrillation recurrence identification system 400 may provide different outcomes dependent upon an atrial fibrillation type and/or a catheter ablation technique. For example, the disclosed atrial fibrillation recurrence identification system 400 may achieve different AUCs (area under curve) for paroxysmal atrial fibrillation and persistent atrial fibrillation. Therefore, the use of clinical data 406 may allow for the disclosed atrial fibrillation recurrence identification system 400 to aid with patient selection and optimization of atrial fibrillation ablation procedures within a treatment plan.

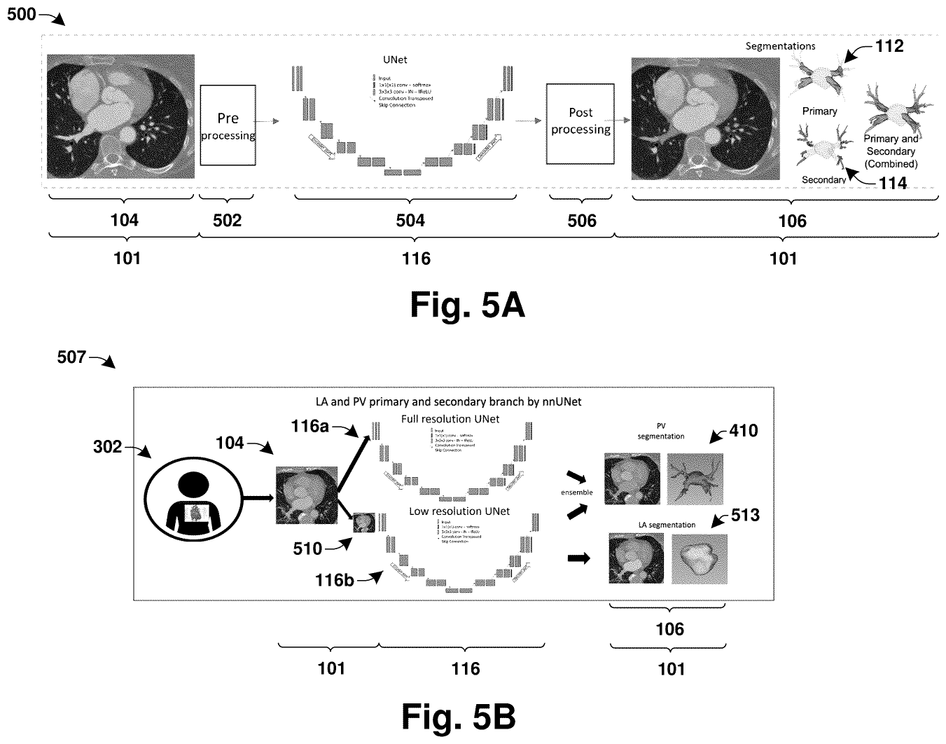

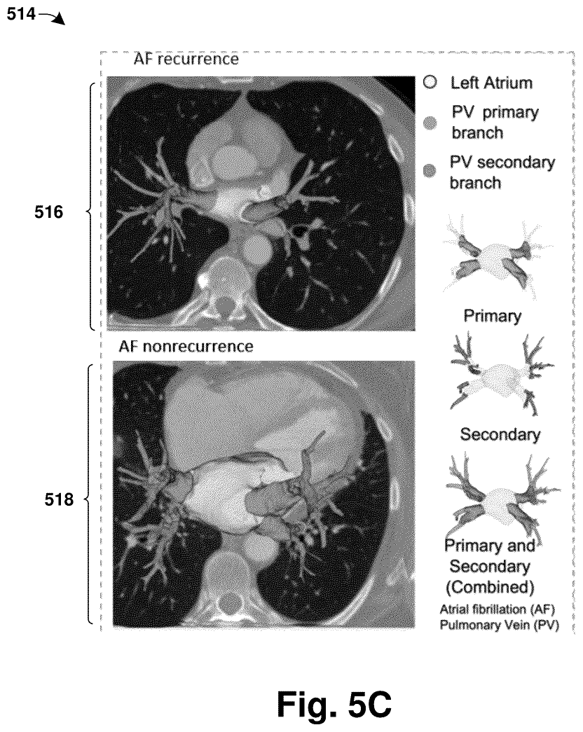

FIG. 5A illustrates some embodiments of a disclosed segmentation process 500 that identifies pulmonary vein branches within digitized imaging data.

The segmentation process 500 comprises a segmentation stage 116 in communication with electronic memory 101 configured to store one or more digitized images 104. The segmentation stage 116 is configured to generate one or more segmented digitized images 106 by identifying one or more ROIs (e.g., primary pulmonary vein branches 112 and/or secondary pulmonary vein branches 114) within the one or more digitized images 104.

In some embodiments, the segmentation stage 116 may comprise a pre-processing stage 502 and a neural network 504. The pre-processing stage 502 is configured to perform one or more pre-processing operations on the one or more digitized images 104. In some embodiments, the pre-processing stage 502 may be configured to perform isotropic resampling of the one or more digitized images 104 at different scales within the primary and secondary pulmonary vein branches. For example, the pre-processing stage 502 may be configured to perform isotropic resampling to a uniform voxel size of 1 mm (millimeter)×1 mm×1 mm in primary pulmonary vein branches and a uniform voxel size of 0.5 mm×0.5 mm×0.5 mm in secondary pulmonary vein branches to keep intricate shape details of the narrow secondary pulmonary vein branches. In some embodiments, the pre-processing stage 502 may be configured to apply Gaussian blurring during resampling to reduce aliasing effects. These preprocessing techniques help to standardize spatial resolution and minimize artifacts induced by differences in slice thickness, thereby enhancing an accuracy of a medical prediction.

In some additional embodiments, the segmentation stage 116 may further comprise a post-processing stage 506 configured to perform one or more post-processing operations. In some embodiments, the one or more post-processing operations may include removal of clearly erroneous regions of a segmented image (e.g., a clear mismatch between the one or more digitized images 104 and a probability map).

FIG. 5B illustrates some additional embodiments of a disclosed segmentation process 507 configured to identify pulmonary vein branches within digitized imaging data.

The segmentation process 507 comprises a segmentation stage 116 in communication with electronic memory 101 configured to store one or more digitized images 104 (e.g., one or more CT scans). The segmentation stage 116 comprises a first neural network 116a (e.g., a full resolution U-Net) having a first resolution and a second neural network 116b (e.g., a low resolution U-Net) having a second resolution that is different than (e.g., less than) the first resolution. The first neural network 116a and the second neural network 116b are both configured to operate upon the one or more digitized images 104 to generate one or more segmented digitized images 106. For example, the first neural network 116a may be configured to perform a first segmentation process to generate first segmentation data (e.g., a first probability map) and the second neural network 116b may be configured to perform a second segmentation process to generate second segmentation data (e.g., a second probability map).

In some embodiments, the first segmentation data and the second segmentation data may be collectively used to generate a composite label map 410. The second segmentation data may be used to generate a second segmented image 513 that identifies the left atrium. It has been appreciated that integrating both low and full resolution networks via an ensemble approach aids in maintaining effective segmentations even in the presence of moderate amounts of image noise. In some embodiments (not shown), the left atrium may be subtracted from the composite label map 410 to generate a pulmonary vein label map that isolates the pulmonary veins in the cardiac anatomy. Lastly, a set of morphologic operations may be applied to remove the secondary pulmonary vein branches from the pulmonary vein label map to isolate the primary pulmonary vein branches.

In some embodiments, the second neural network 116b may be trained on down-sampled (e.g., low-resolution) versions 510 of the one or more digitized images 104 to capture larger context, while the first neural network 116a is trained upon full-resolution versions of the one or more digitized images 104 to capture detailed structures. In some embodiments, an Adam optimizer may be used to train the first neural network 116a and the second neural network 116b (e.g., with a learning rate of 1e-4 and a batch size of 4).

FIG. 5C illustrates some embodiments of an exemplary segmented images 514 identifying one or more regions of interest.

Segmented image 516 is from a patient that has experienced atrial fibrillation recurrence. The segmented image 516 comprises a left atrium, primary pulmonary vein branches, and secondary pulmonary vein branches. The primary pulmonary vein branches extend outward from the left atrium. The secondary pulmonary vein branches extend outward from the primary pulmonary vein branches.

Segmented image 518 is from a patient that has not experienced atrial fibrillation recurrence. The segmented image 518 comprises a left atrium, primary pulmonary vein branches, and secondary pulmonary vein branches. By comparing segmented image 516 and segmented image 518, it can be seen that there are structural differences between the patient that has experienced atrial fibrillation recurrence and the patient that has not experienced atrial fibrillation recurrence. These structural differences can be characterized by the plurality of radiomic features and used by the disclosed atrial fibrillation recurrence identification system to generate a medical prediction of atrial fibrillation recurrence.

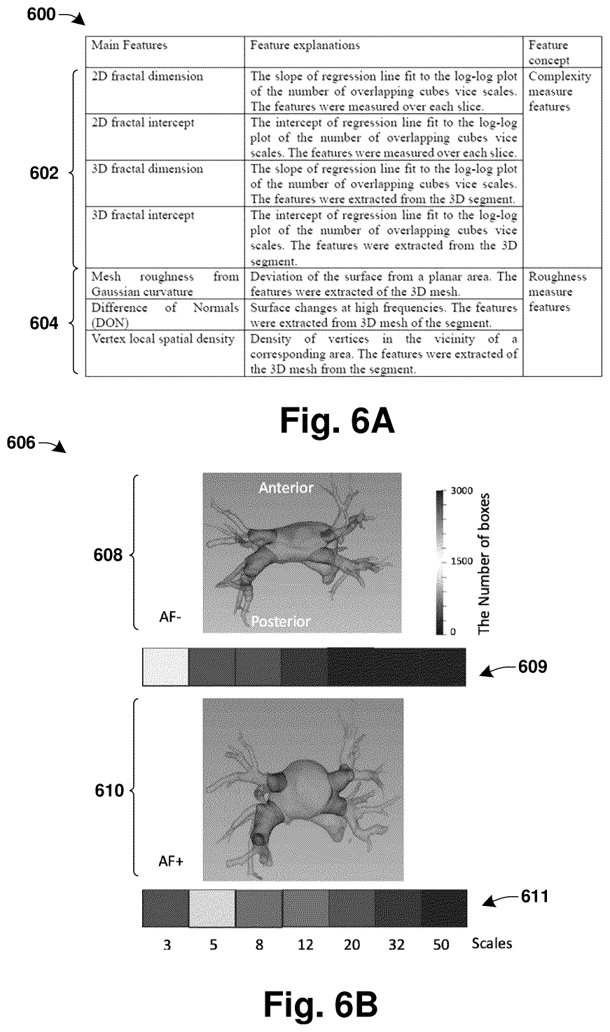

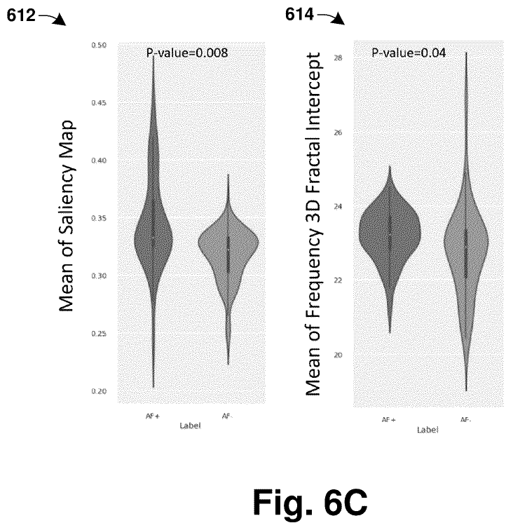

FIG. 6A illustrates a table 600 showing exemplary radiomic features extracted from digitized imaging data. It will be appreciated that the exemplary radiomic features illustrated in table 600 are merely examples of radiomic features that may be used by the disclosed atrial fibrillation recurrence identification system and that other radiomic features may also and/or alternatively be used.

As shown in table 600, the exemplary radiomic features comprise a mixture of fractal-based features 602 and mesh-based features 604. The fractal-based features 602 include two dimensional (2D) fractal dimension features, 2D fractal intercept features, 3D fractal dimension features, and 3D fractal intercept features. The mesh-based features 604 include mesh roughness from Gaussian curvature features, difference of normal (DON) features, and vertex local spatial density features. In some embodiments, the radiomic features may comprise statistical measures (e.g., a mean, a maximum, a variance, a standard deviation, a skewness, etc.) of the radiomic features shown in table 600.

The fractal dimension slope characterizes the anisotropy degree and direction of a measured surface. The fractal dimension slope may comprise a slope of a logarithmic regression line of scale and a number of overlapped grid boxes with a region of interest. The fractal dimension intercept is an intercept of the logarithmic regression line. The mesh roughness from Gaussian Curvature represents a deviation of a surface from a planar area. The DON detects surface changes at high frequencies, especially at edges. To compute vertex local spatial density, a percentage interval of 0.1% to 0.5% of a segment size may be chosen and a number of vertices in a vicinity of a corresponding area may be measured. A maximum number threshold is chosen. If the number exceeds the threshold in each percentage, a score is added to the focused vertex. The score is proportionally correlated with density. Consequently, this method results in a vertex density score based on the global density of the object at multiple sizes.

FIG. 6B illustrates some embodiments of an exemplary segmented image 606 and corresponding heat maps illustrating 3D fractal dimensions.

FIG. 6B illustrates a first cardiac image 608 from a patient that did not experience atrial fibrillation recurrence after an ablation and a second cardiac image 610 from a patient that did experience atrial fibrillation recurrence after an ablation. As can be seen in heat maps, 609 and 611, a higher number of boxes appear across various scales in the second cardiac image 610, indicating greater surface complexity compared to first cardiac image. Therefore, FIG. 6B shows a clear relationship between surface complexity and a likelihood of atrial fibrillation recurrence after an ablation.

FIG. 6C illustrates some embodiments of violin plots of exemplary radiomic features extracted from primary pulmonary vein branches.

The violin plots include a first violin plot 612 corresponding to a statistically significant radiomic feature including a mean of a saliency map and a second violin plot 614 corresponding to a second radiomic feature including a mean of frequency 3D fractal intercept. The first violin plot 612 and the second violin plot 614 respectively include radiomic feature distributions relating to patient's experiencing atrial fibrillation recurrence (AF+) and to patient's experiencing atrial fibrillation non-recurrence (AF−). As shown in the first violin plot 612 and the second violin plot 614, the distributions relating to atrial fibrillation recurrence (AF+) and to atrial fibrillation non-recurrence (AF−) are significantly different, thereby indicating that the first and second radiomic features can be used to generate a medical prediction that accurately predicts atrial fibrillation recurrence.

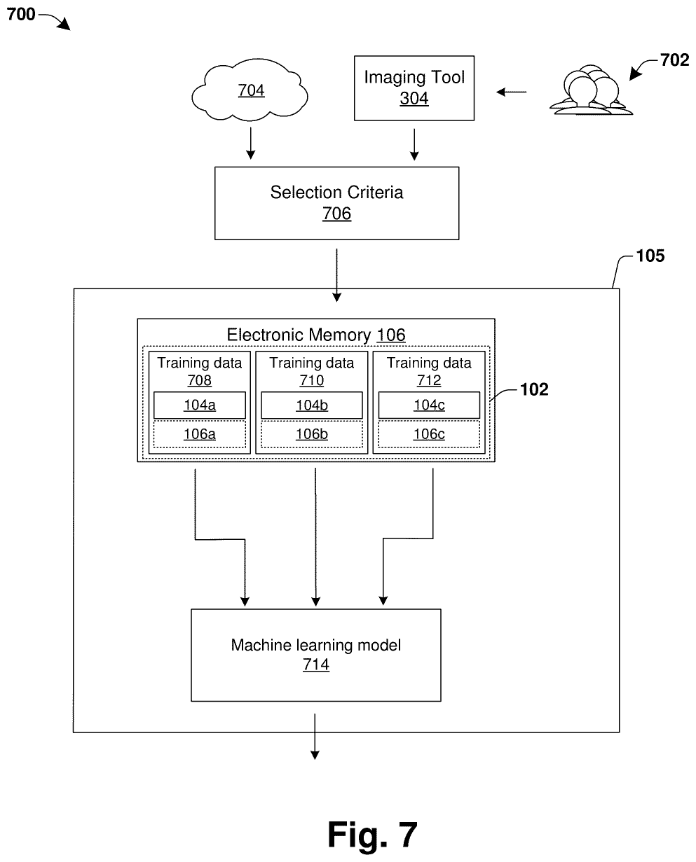

FIG. 7 illustrates some additional embodiments of an atrial fibrillation recurrence identification system 700 comprising a machine learning stage configured to generate a medical prediction corresponding to atrial fibrillation recurrence.

The atrial fibrillation recurrence identification system 700 comprises an electronic memory 101 configured to store digitized imaging data 102 from a plurality of patients 702. The electronic memory 101 is configured to store the digitized imaging data 102 as a plurality of different data sets 708-712. In some embodiments, the plurality of different data sets 708-712 may respectively comprise one or more digitized images and/or one or more segmented images. The digitized imaging data 102 may include images from a number of different institutions (e.g., different hospitals), obtained from different scanners, and/or comprising different populations (e.g., female, male, African American, Asian, white, etc.). The digitized imaging data 102 may include images from both patients that have experienced atrial fibrillation recurrence and from patients that have not experienced atrial fibrillation recurrence. The digitized imaging data 102 may include pre-ablation images and/or post-ablation images. In some embodiments, to address an imbalance in a number of atrial fibrillation recurrence and atrial fibrillation non-recurrence cases in different data sets, a synthetic minority over-sampling (SMOTE) technique may be used.

The plurality of different data sets 708-712 may include a training data set 708, a testing data set 710, and/or a validation data set 712. The digitized imaging data 102 may comprise one or more digitized images received from an imaging tool 304 (e.g., a CT scanner) operated upon one or more of the plurality of patients 702 and/or downloaded from an online database 704 (e.g., an online archive). The training data set 708, the testing data set 710, and/or the validation data set 712 may respectively comprise digitized images 104a-104c and/or segmented digitized images 106a-106c relating to a subset of the plurality of patients 702.

In some embodiments, the digitized imaging data 102 obtained from the imaging tool 304 and/or from the online database 704 may be subjected to one or more selection criteria 706 prior to being stored within the plurality of different data sets 708-712. The one or more selection criteria 706 may exclude images that contain unidentifiable pulmonary vein branches, display motion and/or scanning artifacts, have inadequate contrast, have improper contrast timing, have flawed acquisition techniques, and/or the like.

The training data set 708, the testing data set 710, and/or the validation data set 712 may be used to train and validate one or more downstream machine learning models 714. In various embodiments, the one or more downstream machine learning models 714 may include one or more of a segmentation stage (e.g., segmentation stage 116), a feature extraction stage (e.g., feature extraction stage 118), and/or a machine learning stage (e.g., machine learning stage 126).

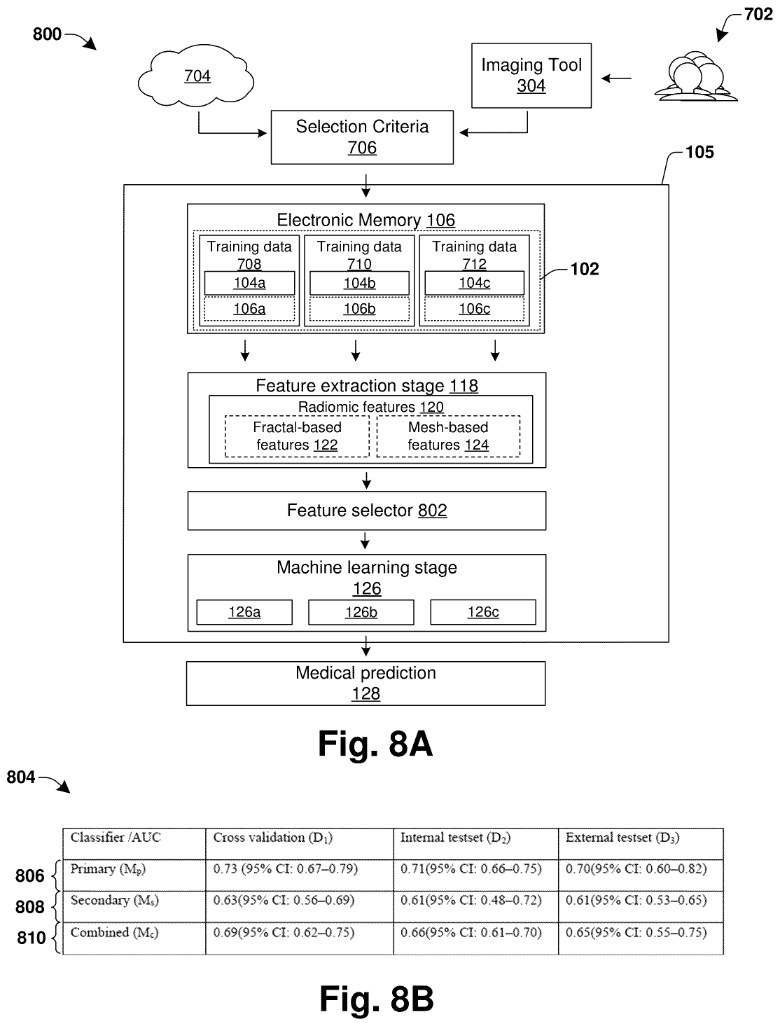

FIG. 8A illustrates some additional embodiments of an atrial fibrillation recurrence identification system 800 comprising a machine learning stage configured to generate a medical prediction corresponding to atrial fibrillation recurrence.

The atrial fibrillation recurrence identification system 800 comprises a feature extraction stage 118 configured to extract a plurality of radiomic features 120 from digitized imaging data 102. The plurality of radiomic features 120 include fractal-based features 122 and mesh-based features 124. In some embodiments, the atrial fibrillation recurrence identification system 800 comprises a feature selector 802 configured to select prognostic features from the plurality of radiomic features 120. The prognostic features are features that are determinative of atrial fibrillation recurrence. In some embodiments, the feature selector 802 may be configured to perform feature selection using an Anova feature selection method. In other embodiments, the feature selector 802 may be configured to perform feature selection using Pearson's correlation coefficient, Chi-square tests, a LASSO regression, and/or the like.

For primary pulmonary vein branches, the feature selector 802 be configured to select more of the fractal-based features 122 as being predictive of atrial fibrillation recurrence than the mesh-based features 124. In some embodiments, the feature selector 802 may select the prognostic features to include one or more of a mean spatial 2D fractal dimension and a mean spatial 3D fractal dimension. This may be because patients that experience atrial fibrillation recurrence have a more irregular structural pattern and surface complexity within the primary branch pulmonary vein than patients that experience atrial fibrillation non-recurrence.

In some embodiments, the machine learning stage 126 may comprise a plurality of different classifiers 126a-126c that have been trained upon radiomic features extracted from different parts of an image. For example, the plurality of different classifiers 126a-126c may comprise a first classifier 126a that has been trained to generate a medical prediction 128 based upon top prognostic fractal-based features and mesh-based features of primary pulmonary vein branches (and not of secondary pulmonary vein branches or a left atrium). In some embodiments, the plurality of different classifiers 126a-126c may further comprise a second classifier 126b that has been trained to generate a medical prediction 128 based upon top prognostic fractal-based features and mesh-based features of the secondary pulmonary vein branches (and not of primary pulmonary vein branches or a left atrium). In some embodiments, the plurality of different classifiers 126a-126c may further comprise a third classifier 126c that has been trained to generate a medical prediction 128 based upon the top prognostic fractal-based features and mesh-based features of both the primary pulmonary vein branches and the secondary pulmonary vein branches (and not a left atrium).

FIG. 8B illustrates a table 804 showing performance parameters of different models within the atrial fibrillation recurrence identification system of FIG. 8A.

The table 804 illustrates performance parameters for a first classifier 806 trained on radiomic features extracted from primary pulmonary vein branches, a second classifier 808 trained on radiomic features extracted from secondary pulmonary vein branches, and a third classifier 810 trained on a combination of radiomic features extracted from primary pulmonary vein branches and secondary pulmonary vein branches.

As shown in FIG. 8B, based on DeLong test statistical analysis, the first classifier 806 consistently and significantly (e.g., p-value<0.05) outperforms the second classifier 808 over multiple evaluation sets, achieving higher AUC (area under curve) values in atrial fibrillation recurrence prediction. This indicates that classifiers trained on radiomic features extracted from secondary pulmonary vein branches yield a lower performance in terms of association with atrial fibrillation recurrence compared to classifiers trained on radiomic features extracted from primary pulmonary vein branches. The first classifier 806 also statistically significantly (e.g., having a p-value<0.05) exhibits superior performance compared to the third classifier 810 on a holdout set (D₂) and external test sets (D₃). Table 804 demonstrates that radiomic features extracted from primary pulmonary vein branches may provide for superior discriminative power compared to radiomic features extracted from secondary pulmonary vein branches.

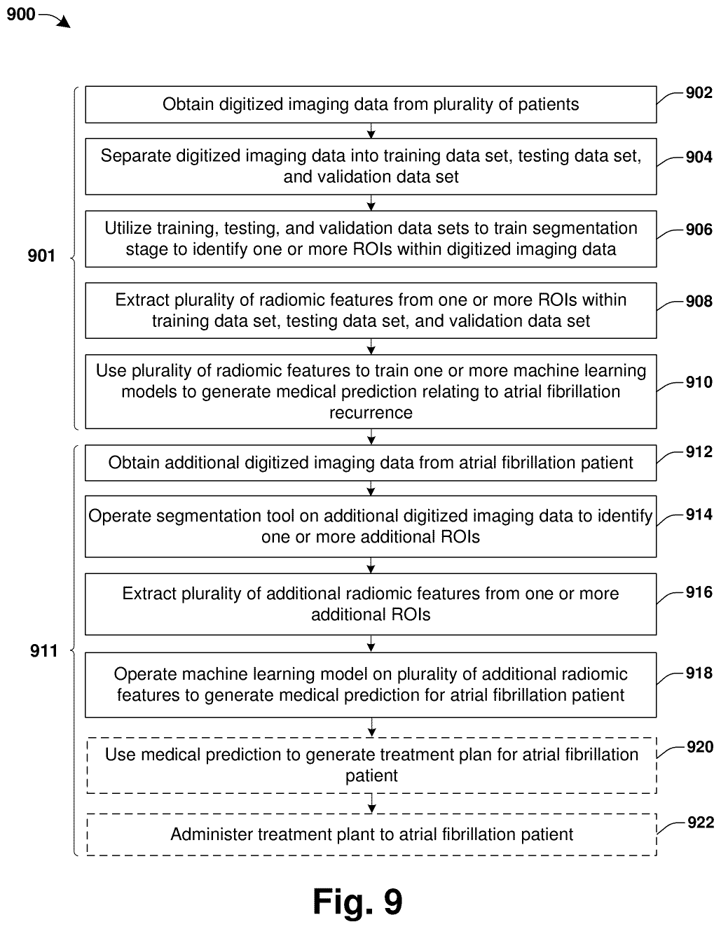

FIG. 9 illustrates a flow diagram showing some additional embodiments of a method 900 of operating a machine learning stage on radiomic features to generate a medical prediction corresponding to atrial fibrillation recurrence.

The method 900 comprises a training phase 901 and an application phase 911. The training phase 901 is configured to train one or more machine learning models to generate a medical prediction corresponding to atrial fibrillation recurrence. In some embodiments, the training phase 901 may be performed according to acts 902-910.

At act 902, digitized imaging data is obtained from a plurality of patients. In some embodiments, the digitized imaging data may comprise a plurality computed tomography (CT) images.

At act 904, the digitized imaging data is separated into a training data set, a testing data set, and a validation data set.

At act 906, the training data set, the testing data set, and the validation data set are used to train a segmentation tool to identify one or more regions of interest (ROIs) within the digitized imaging data.

At act 908, a plurality of radiomic features are extracted from one or more regions of interest within the training data set, the testing data set, and the validation data set.

At act 910, the plurality of radiomic features are used to train one or more machine learning models to generate a medical prediction corresponding to atrial fibrillation recurrence.

The application phase 911 is configured to utilize the one or more trained machine learning models to generate a medical prediction corresponding to atrial fibrillation recurrence for an additional patient. In some embodiments, the application phase 911 may be performed according to acts 912-922.

At act 912, additional digitized imaging data is obtained from an atrial fibrillation patient.

At act 914, the segmentation tool is operated on the additional digitized imaging data to identify one or more additional regions of interest (ROIs).

At act 916, a plurality of additional radiomic features are extracted from the one or more additional ROIs.

At act 918, the one or more machine learning models are operated on the plurality of additional radiomic features to generate a medical prediction corresponding to atrial fibrillation recurrence for the atrial fibrillation patient.

At act 920, the medical prediction may be used to generate a treatment plan for the atrial fibrillation patient. The treatment plan may include application of drugs (e.g., beta-blockers, flecainide, propafenone, amiodarone, etc.), medical procedures (e.g., repeating catheter ablation, electrical cardioversion, etc.), making lifestyle changes to manage underlying risk factors like high blood pressure and obesity, etc.

At act 922, the treatment plan may be administered to the atrial fibrillation patient by a health care professional.

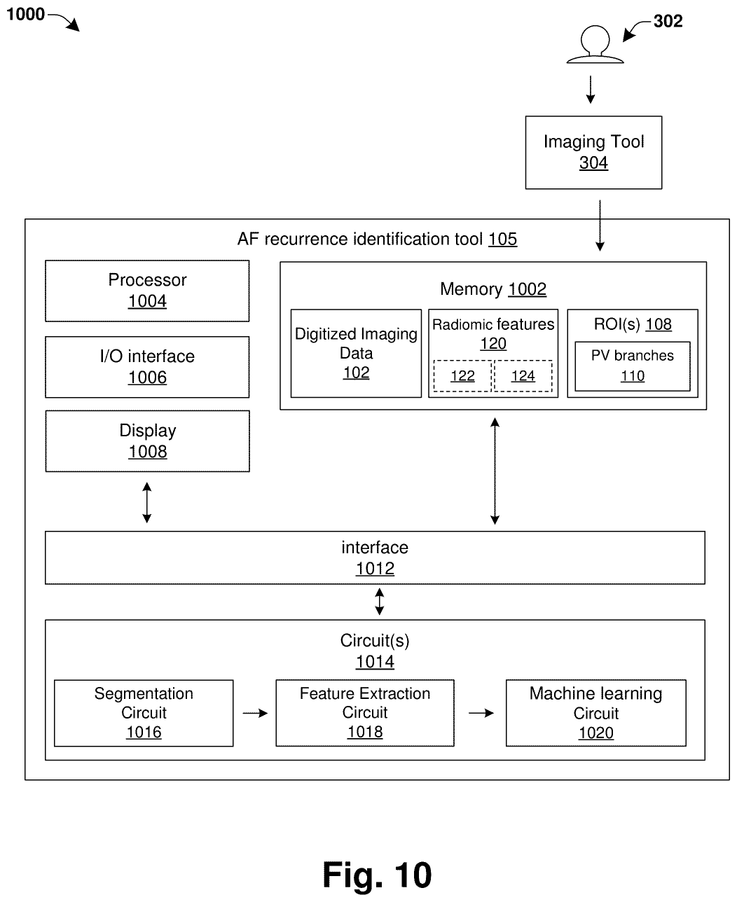

FIG. 10 illustrates a block diagram of some embodiments of an atrial fibrillation recurrence identification system 1000 comprising a machine learning stage configured to generate a medical prediction corresponding to atrial fibrillation.

The atrial fibrillation recurrence identification system 1000 comprises an atrial fibrillation recurrence identification tool 105. The atrial fibrillation recurrence identification tool 105 is coupled to an imaging tool 304 (e.g., a CT imaging tool) that is configured to generate digitized imaging data 102 (e.g., one or more digitized images) of a patient 302. In some embodiments, the imaging tool 304 may comprise a computed tomography scanner.

The atrial fibrillation recurrence identification tool 105 comprises a processor 1004 and a memory 1002. The processor 1004 can, in various embodiments, comprise circuitry such as, but not limited to, one or more single-core or multi-core processors. The processor 1004 can include any combination of general-purpose processors and dedicated processors (e.g., graphics processors, application processors, etc.). The processor(s) 1004 can be coupled with and/or can comprise memory (e.g., memory 1002) or storage and can be configured to execute instructions stored in the memory 1002 or storage to enable various apparatus, applications, or operating systems to perform operations and/or methods discussed herein. The memory 1002 can be further configured to store digitized imaging data 102 comprising the one or more digitized images (e.g., CT images) obtained by the imaging tool 304. The one or more digitized images 104 may comprise respectively a plurality of pixels, each pixel having an associated intensity.

The atrial fibrillation recurrence identification tool 105 also comprises an input/output (I/O) interface 1006 (e.g., associated with one or more I/O devices), a display 1008, one or more circuits 1014, and an interface 1012 that connects the processor 1004, the memory 1002, the I/O interface 1006, the display 1008, and the one or more circuits 1014. The I/O interface 1006 can be configured to transfer data between the memory 1002, the processor 1004, the one or more circuits 1014, and external devices (e.g., the CT imaging tool).

In some embodiments, the one or more circuits 1014 may comprise hardware components. In other embodiments, the one or more circuits 1014 may comprise software components. The one or more circuits 1014 can comprise a segmentation circuit 1016 (e.g., a deep learning circuit) configured to perform a segmentation operation on one or more digitized images within the digitized imaging data 102 to identify one or more regions of interest (ROIs) 108 comprising pulmonary vein branches 110 (e.g., primary pulmonary veins, secondary pulmonary veins).

In some additional embodiments, the one or more circuits 1014 may further comprise feature extraction circuit 1018 configured to extract a plurality of radiomic features 120 from the one or more regions of interest (ROIs) 108. The plurality of radiomic features 120 characterize a structural complexity and/or roughness of the pulmonary vein branches. The plurality of radiomic features 120 may be stored in the memory 1002. In some embodiments, the plurality of radiomic features 120 may comprise fractal-based features 122 and mesh-based features 124.

In some embodiments, the one or more circuits 1014 may further comprise a machine learning circuit 1020 configured to operate one or more machine learning models (e.g., a gradient boosting classifier) upon the plurality of radiomic features 120 to generate a medical prediction 128 corresponding to atrial fibrillation recurrence.

Embodiments discussed herein relate to training and/or employing machine learning models (e.g., unsupervised (e.g., clustering) or supervised (e.g., classifiers, etc.) models) to determine a medical prediction based on a combination of radiomic features and deep learning, based at least in part on radiomic features of medical imaging scans (e.g., MRI, CT, etc.) that are not perceivable by the human eye, and involve computation that cannot be practically performed in the human mind. As one example, machine learning classifiers and/or deep learning models as described herein cannot be implemented in the human mind or with pencil and paper. Embodiments thus perform actions, steps, processes, or other actions that are not practically performed in the human mind, at least because they require a processor or circuitry to access digitized images stored in a computer memory and to extract or compute radiomic features that are based on the digitized images and not on properties of tissue or the images that are perceivable by the human eye. Embodiments described herein can use a combined order of specific rules, elements, operations, or components that render information into a specific format that can then be used and applied to create desired results more accurately, more consistently, and with greater reliability than existing approaches, thereby producing the technical effect of improving the performance of the machine, computer, or system with which embodiments are implemented.

### Example Use Case

Background: Atrial fibrillation (AF) recurrence is common after catheter ablation. Pulmonary vein (PV) isolation is the cornerstone of AF ablation, but pulmonary vein remodeling may be associated with the risk of AF recurrence. We aimed to evaluate whether artificial intelligence (AI)-based morphological features of primary and secondary pulmonary vein branches on CT images are associated with AF recurrence post ablation.

Methods: Two AI models were trained for segmentation of CT images, enabling isolation of pulmonary vein branches. Patients from Cleveland Clinic (n=135) and Vanderbilt University (n=594) were combined and divided into two sets for training and cross validation (D₁,n=218), and internal testing (D₂,n=511). An independent validation set (D₃,n=80) was obtained from University Hospitals of Cleveland. We extracted 48 fractal-based and 12 shape-based radiomic features from primary and secondary pulmonary vein branches of patients with AF recurrence (AF+) and without recurrence (AF−) after catheter ablation of AF. To predict AF recurrence, three Gradient Boosting classification models based on significant features from primary (Mₚ), secondary (Mₛ), and combined (M_{c}) pulmonary vein branches were built.

Results: Features relating to primary PVs were found to be associated with AF recurrence. The Mₚ classifier achieved AUC values of 0.73, 0.71, and 0.70 across the three datasets. AF+ cases exhibited greater surface complexity in their primary pulmonary vein area, as evidenced by higher fractal dimension values compared to AF-cases. The Mₛ classifier results revealed weaker association with AF recurrence, suggesting higher relevance to AF recurrence post-ablation from primary pulmonary vein branch morphology.

Conclusions: This largest multi-institutional study to date revealed associations between AI-extracted morphological features of the primary pulmonary vein branches with AF recurrence in 809 patients from three sites. Future work will focus on enhancing the predictive ability of the classifier by integrating clinical, structural, and morphological features, including LAA and LA-related characteristics.

Therefore, the present disclosure provides a method and apparatus that uses radiomic features extracted from pulmonary vein branches to generate a medical prediction corresponding to atrial fibrillation recurrence.

In some embodiments, the present disclosure relates to a method, including accessing digitized imaging data stored in an electronic memory, the digitized imaging data comprising one or more regions of interest including pulmonary vein branches of a patient; extracting a plurality of radiomic features from the one or more regions of interest, the plurality of radiomic features including one or more of fractal-based features or mesh-based features; and providing the plurality of radiomic features to a machine learning stage, the machine learning stage being configured to utilize the plurality of radiomic features to generate a medical prediction corresponding to atrial fibrillation recurrence after an ablation procedure.

In other embodiments, the present disclosure relates to a non-transitory computer-readable medium storing computer-executable instructions that, when executed, cause a processor to perform operations, including accessing digitized imaging data, the digitized imaging data identifying one or more regions of interest including one or more primary pulmonary vein branches of a patient prior to an ablation procedure; extracting a plurality of radiomic features from the one or more regions of interest, the plurality of radiomic features characterizing morphological attributes associated with the one or more primary pulmonary vein branches; and operating upon the plurality of radiomic features with a machine learning stage, the machine learning stage being configured to utilize the plurality of radiomic features to generate a medical prediction corresponding to atrial fibrillation recurrence in response to the ablation procedure

In yet other embodiments, the present disclosure relates to an apparatus, including an electronic memory configured to store digitized imaging data including one or more segmented digitized images that identify one or more regions of interest including pulmonary vein branches of a patient; a feature extraction stage configured to extract a plurality of features from the one or more regions of interest, the plurality of features including fractal-based features and mesh-based features that characterize morphological attributes associated with the pulmonary vein branches; and a machine learning stage configured to utilize the plurality of features to generate a medical prediction corresponding to atrial fibrillation recurrence.

Examples herein can include subject matter such as an apparatus, a digital whole slide scanner, a CT system, an MRI system, a personalized medicine system, a CADx system, a processor, a system, circuitry, a method, means for performing acts, steps, or blocks of the method, at least one machine-readable medium including executable instructions that, when performed by a machine (e.g., a processor with memory, an application-specific integrated circuit (ASIC), a field programmable gate array (FPGA), or the like) cause the machine to perform acts of the method or of an apparatus or system according to embodiments and examples described.

References to “one embodiment”, “an embodiment”, “one example”, and “an example” indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in one embodiment” does not necessarily refer to the same embodiment, though it may.

“Computer-readable storage device”, as used herein, refers to a device that stores instructions or data. “Computer-readable storage device” does not refer to propagated signals. A computer-readable storage device may take forms, including, but not limited to, non-volatile media, and volatile media. Non-volatile media may include, for example, optical disks, magnetic disks, tapes, and other media. Volatile media may include, for example, semiconductor memories, dynamic memory, and other media. Common forms of a computer-readable storage device may include, but are not limited to, a floppy disk, a flexible disk, a hard disk, a magnetic tape, other magnetic medium, an application specific integrated circuit (ASIC), a compact disk (CD), other optical medium, a random access memory (RAM), a read only memory (ROM), a memory chip or card, a memory stick, and other media from which a computer, a processor or other electronic device can read.

“Circuit”, as used herein, includes but is not limited to hardware, firmware, software in execution on a machine, or combinations of each to perform a function(s) or an action(s), or to cause a function or action from another logic, method, or system. A circuit may include a software controlled microprocessor, a discrete logic (e.g., ASIC), an analog circuit, a digital circuit, a programmed logic device, a memory device containing instructions, and other physical devices. A circuit may include one or more gates, combinations of gates, or other circuit components. Where multiple logical circuits are described, it may be possible to incorporate the multiple logical circuits into one physical circuit. Similarly, where a single logical circuit is described, it may be possible to distribute that single logical circuit between multiple physical circuits.

To the extent that the term “includes” or “including” is employed in the detailed description or the claims, it is intended to be inclusive in a manner similar to the term “comprising” as that term is interpreted when employed as a transitional word in a claim.

Throughout this specification and the claims that follow, unless the context requires otherwise, the words ‘comprise’ and ‘include’ and variations such as ‘comprising’ and ‘including’ will be understood to be terms of inclusion and not exclusion. For example, when such terms are used to refer to a stated integer or group of integers, such terms do not imply the exclusion of any other integer or group of integers.

To the extent that the term “or” is employed in the detailed description or claims (e.g., A or B) it is intended to mean “A or B or both”. When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995).

While example systems, methods, and other embodiments have been illustrated by describing examples, and while the examples have been described in considerable detail, it is not the intention of the applicants to restrict or in any way limit the scope of the appended claims to such detail. It is, of course, not possible to describe every conceivable combination of components or methodologies for purposes of describing the systems, methods, and other embodiments described herein. Therefore, the invention is not limited to the specific details, the representative apparatus, and illustrative examples shown and described. Thus, this application is intended to embrace alterations, modifications, and variations that fall within the scope of the appended claims.

What is claimed is:

1. A method, comprising:
accessing digitized imaging data stored in an electronic memory, the digitized imaging data comprising one or more regions of interest including pulmonary vein branches of a patient;
extracting a plurality of radiomic features from the one or more regions of interest, wherein the plurality of radiomic features comprise one or more of fractal-based features or mesh-based features; and
providing the plurality of radiomic features to a machine learning stage, wherein the machine learning stage is configured to utilize the plurality of radiomic features to generate a medical prediction corresponding to atrial fibrillation recurrence after an ablation procedure.

2. The method of claim 1, wherein the pulmonary vein branches comprise primary pulmonary vein branches extending outward from a left atrium of a heart and secondary pulmonary vein branches extending outward from the primary pulmonary vein branches.

3. The method of claim 1, further comprising:
operating a segmentation tool to segment the digitized imaging data to identify the one or more regions of interest, wherein the segmentation tool comprises a plurality of neural networks having different resolutions; and
wherein the plurality of neural networks are respectively configured to generate probability maps that are combined to identify the one or more regions of interest.

4. The method of claim 1, wherein the pulmonary vein branches consist of primary pulmonary vein branches extending outward from a heart.

5. The method of claim 1, wherein the fractal-based features are measured in both a spatial domain and a frequency domain.

6. The method of claim 1, wherein the fractal-based features include two-dimensional (2D) fractal dimension features, 2D fractal intercept features, three-dimensional (3D) fractal dimension features, and 3D fractal intercept features.

7. The method of claim 1, further comprising:
operating an imaging tool to generate one or more digitized images of the patient, wherein the digitized imaging data includes segmented versions of the one or more digitized images.

8. The method of claim 1, wherein the mesh-based features measure a roughness of the pulmonary vein branches.

9. The method of claim 1, further comprising:
generating a treatment plan based on the medical prediction; and
applying the treatment plan to the patient.

10. The method of claim 1, further comprising:
providing clinical data to the machine learning stage, wherein the machine learning stage is configured to utilize the plurality of radiomic features and the clinical data to generate the medical prediction.

11. The method of claim 1, further comprising:
identifying a target region of the pulmonary vein branches using the plurality of radiomic features, wherein the target region comprises a region of tissue that is targeted during the ablation procedure to mitigate atrial fibrillation recurrence.

12. The method of claim 1, wherein the digitized imaging data includes pre-ablation imaging data.

13. A non-transitory computer-readable medium storing computer-executable instructions that, when executed, cause a processor to perform operations, comprising:
accessing digitized imaging data, the digitized imaging data identifying one or more regions of interest including one or more primary pulmonary vein branches of a patient prior to an ablation procedure;
extracting a plurality of radiomic features from the one or more regions of interest, wherein the plurality of radiomic features characterize morphological attributes associated with the one or more primary pulmonary vein branches; and
operating upon the plurality of radiomic features with a machine learning stage, wherein the machine learning stage is configured to utilize the plurality of radiomic features to generate a medical prediction corresponding to atrial fibrillation recurrence in response to the ablation procedure.

14. The non-transitory computer-readable medium of claim 13, wherein the one or more regions of interest further include one or more secondary pulmonary vein branches.

15. The non-transitory computer-readable medium of claim 13, further comprising:
generating a composite label map comprising a left atrium, the one or more primary pulmonary vein branches, and secondary pulmonary vein branches;
generating a pulmonary vein label map by subtracting the left atrium from the composite label map; and
identifying the one or more primary pulmonary vein branches by removing the secondary pulmonary vein branches from the pulmonary vein label map by morphologic operations.

16. An apparatus, comprising:
an electronic memory configured to store digitized imaging data comprising one or more segmented digitized images that identify one or more regions of interest including pulmonary vein branches of a patient;
a feature extraction stage configured to extract a plurality of features from the one or more regions of interest, wherein the plurality of features comprise fractal-based features and mesh-based features that characterize morphological attributes associated with the pulmonary vein branches; and
a machine learning stage configured to utilize the plurality of features to generate a medical prediction corresponding to atrial fibrillation recurrence.

17. The apparatus of claim 16, further comprising:
a segmentation stage configured to segment one or more digitized images to identify the one or more regions of interest.

18. The apparatus of claim 17, wherein the segmentation stage comprises:
a first neural network having a first resolution and a second neural network having a second resolution, which is different than the first resolution, wherein the one or more regions of interest are identified using outputs from both the first neural network and the second neural network.

19. The apparatus of claim 16, wherein the one or more regions of interest comprise primary pulmonary vein branches, but not secondary vein branches.

20. The apparatus of claim 16, wherein the one or more regions of interest comprise primary pulmonary vein branches, but not a left atrium.
